(19) United States
(12) Patent Application Publication
(10) Pub. No.: US 2011/0039307 A1
(43) Pub. Date: Feb. 17, 2011

(54) ETHANOL YIELDS IN FERMENTATION FROM AN IMPROVED LIQUEFACTION PROCESS

(76) Inventors: Jodi M. HENDERSON, Roscoe, IL (US); Randall J. DOYAL, Owatonna, MN (US)

Correspondence Address: DANISCO US INC.;ATTENTION: LEGAL DEPARTMENT, 925 PAGE MILL ROAD, PALO ALTO, CA, 94304, US

(21) Appl. No.: 12/778,393

(22) Filed: May 12, 2010

Related U.S. Application Data
(60) Provisional application No. 61/177,428, filed on May 12, 2009.

(51) Int. Cl. C12P 19/14; C12P 7/06

(52) U.S. Cl. 435/99; 435/161

(57) ABSTRACT

An improved liquefaction process comprises the use of an increased dose of alpha-amylase coupled with a liquefaction temperature no higher than 99° C., e.g., in the range of about 88-92° C. The improved liquefaction process advantageously omits the conventional high-temperature treatment, e.g., jet-cooking at a temperature of about 95-125° C., so that starch can be processed more economically into ethanol, for example. In one embodiment, the improved liquefaction process results in an increased yield of ethanol using commercially available alpha-amylases.

### CROSS-REFERENCE TO RELATED APPLICATIONS

This application claims the benefit of U.S. Provisional Patent Application Ser. No. 61/177,428, filed on May 12, 2009, the contents of which are incorporated herein by reference in its entirety.

### FIELD OF THE INVENTION

Described herein is a process of liquefying starch for producing fermentation products, for example, ethanol. The disclosure also provides a process for producing ethanol comprising liquefying starch in accordance with the liquefaction process described herein.

### BACKGROUND

The conversion of vegetable starches, especially cornstarch, to ethanol is a rapidly expanding industry. Ethanol has widespread applications as an industrial chemical, a gasoline additive, or a liquid fuel by itself. The use of ethanol as a fuel or fuel additive significantly reduces air emissions while maintaining or even improving engine performance. On the other hand, ethanol is a renewable fuel, so that its use may reduce dependence on finite fossil fuel sources. Furthermore, use of ethanol may decrease the net accumulation of carbon dioxide in the atmosphere.

A typical process of ethanol production from starch-containing raw materials comprises two sequential enzyme-catalyzed steps that result in the production of glucose. Yeast is then used to ferment the glucose to ethanol. The first step is starch liquefaction catalyzed by alpha-amylases. Alpha-amylases (EC 3.2.1.1) are endohydrolases that catalyze the random cleavage of internal α-1,4-D-glucosidic bonds. They are capable of degrading the viscous liquefact to maltodextrins. As alpha-amylases break down the starch, the viscosity decreases. Because liquefaction typically is conducted at high temperatures to break down the starch granules, thermostable alpha-amylases, such as an alpha-amylase from Bacillus sp., are typically used. Enzymatic liquefaction is often carried out in a multi-step process. After the enzyme(s) is (are) added, the slurry is heated to a temperature between about 60-95° C., typically about 78-88° C. Subsequently, the slurry is heated, for example jet-cooked or otherwise, to a temperature typically between about 95-125° C., and then cooled to about 60-95° C. More enzyme(s) is (are) added, and the mash is held for another about 0.5-4 hours at the desired temperature, typically about 60-95° C., to obtain the final hydrolysis reaction. In another frequently used liquefaction process, the slurry supplemented with enzyme(s) is first heated to a temperature between about 60-95° C., typically about 78-88° C. The mash is liquefied at this temperature for about 0.5-4 hours. Subsequently, the mash is heated, for example jet-cooked or otherwise, to a temperature typically between about 95-125° C. to achieve desired hydrolysis.

The maltodextrins produced in this manner generally cannot be fermented by yeast to form alcohol. A second enzyme-catalyzed saccharification step thus is required to break down the maltodextrins. Glucoamylases and/or maltogenic alpha-amylases commonly are used to catalyze the hydrolysis of non-reducing ends of the maltodextrins formed after liquefaction to release D-glucose, maltose and isomaltose. Debranching enzymes, such as pullulanases, can be used to aid saccharification. Saccharification typically takes place under acidic conditions at elevated temperatures, e.g., about 60° C., pH 4.3.

A typical saccharification reaction used to produce various products is depicted below:

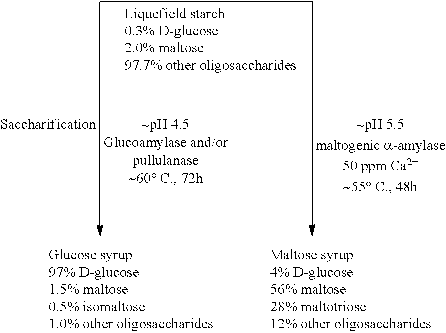

While enzymatic starch liquefaction processes are well established, there is a need for further improvement for commercial starch processing, especially in ethanol production. In particular, there is an ongoing need to improve the efficiency of converting starch to ethanol and reduce the overall energy required to make ethanol. Liquefaction typically requires high temperatures for heating the starch slurry, e.g., jet-cooking, to break down the starch granule. In addition to a high-energy demand, the high-temperature liquefaction results in products that are subsequently unable to be fermented to desired products, such as ethanol, thereby reducing the potential yield. Particularly, fermentable sugar can be lost during high-temperature treatments due to the Maillard reaction, which is a chemical reaction between an amino acid and a reducing sugar, typically requiring heat. The reaction between the reactive carbonyl group of sugar and the nucleophilic amino group of an amino acid forms a variety of poorly characterized molecules, resulting a lower yield of fermentable sugars. Accordingly, providing a liquefaction process at reduced temperatures would conserve energy, making ethanol production more economical and environmentally beneficial.

Increases of ethanol yield have been achieved by modifying the liquefaction process. See e.g., US 2007/0141689, published on Jun. 21, 2007; US 2007/0184150, published on Aug. 9, 2007; US 2008/0009048, published on Jan. 10, 2007; and US 2008/0121227, published on May 29, 2008. The modified liquefaction processes may include, (1) an alpha-amylase treatment performed first at a lower temperature and transferred then to a higher temperature; (2) the addition of a pullulanase; (3) supplementation of a fungal acid alpha-amylase to reach a higher DE value; and/or (4) a longer liquefaction time. These modifications are onerous, however, because they demand additional adjustments of the liquefaction process, e.g., shifting between multiple temperatures or adjusting pH values to accommodating various enzymes. Therefore, there is a need for liquefaction processes that are less burdensome while more efficient or economical in producing ethanol.

### SUMMARY

An improved liquefaction process by utilizing a higher than normal alpha-amylase dose advantageously eliminates the high temperature liquefaction step(s), matches the overall processing time of the high-temperature liquefaction, and improves the yields as well as the overall economics of ethanol production. The elimination of high temperature step(s) may also conserve energy, making ethanol production more economical and environmentally beneficial. For reference, even a 1% increase in ethanol yield will result in a $750,000 increase in revenues for a 50 million gallon per year (MM gpy) plant when ethanol is sold at $1.50/gallon.

An improved method of processing a starch accordingly comprises liquefying starch at a lower temperature in the presence of an increased dose of alpha-amylase. The increased dose of alpha-amylase may be at least about 1.7, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, or about 10.0 time of the amount of alpha-amylase that is required to reach a DE value of at least about 10 within 90 minutes in a liquefaction process performed at a temperature of about 85° C. The improved liquefying method can be performed at a temperature no higher than 99° C., e.g., in a range of about 70° C. to about 95° C., about 80° C. to about 95° C., about 85° C. to about 95° C., or optionally about 88° C. to about 92° C. The liquefaction may last about 30-300 minutes, e.g., 30-180 minutes. At the end of the liquefying, there is residual alpha-amylase activity, which may be useful in downstream processes. The residual alpha-amylase activity of at least about 10%, e.g., about 11%, about 12%, about 13%, about 14% or about 15%, may be present after liquefying the starch.

The present starch processing method may further comprise saccharifying liquefied starch and fermenting saccharified starch to produce ethanol. Another aspect contemplates a method comprising recovering the ethanol. The ethanol production may further comprise distilling the ethanol. Fermenting and distilling may be carried out simultaneously, separately, or sequentially.

One aspect of the present disclosure contemplates a more efficient conversion of ethanol from starch. At the end of ethanol production, the residual starch present in 100 gram of grain by-product is at least about 10%, about 20%, or about 30% lower than that of an ethanol production process having starch liquefied at a temperature of about 85° C. and at a alpha-amylase dosage required to reach a DE value of at least about 10 within 90 minutes. A further aspect of the present disclosure contemplates producing ethanol at a higher yield. The ethanol yield, e.g., gallon of undenatured ethanol per bushel of corn, may be at least about 1.0%, about 1.5%, about 2.0%, about 2.5%, or about 3.0% higher than that of an ethanol production process having starch liquefied at a temperature of about 85° C. and at a alpha-amylase dosage required to reach a DE value of at least about 10 within 90 minutes.

A further aspect contemplates a starch processing method that is suitable for starch from a group consisting of corns, cobs, wheat, barley, rye, milo, and potatoes, and any combination of these. Typically, the starch is from corn or corn mash.

### INCORPORATION BY REFERENCE

All publications and patent applications mentioned in this specification are herein incorporated by reference to the same extent as if each publication or patent application was specifically and individually indicated to be incorporated by reference.

### DETAILED DESCRIPTION

An improved liquefaction process comprises the use of an increased dose of alpha-amylase coupled with a liquefaction temperature no higher than 99° C. The improved liquefaction process advantageously omits the jet-cooking or conventional high-temperature treatment, so that starch-containing raw materials can be processed more efficiently and economically into ethanol, for example. Particularly, the improved liquefaction process requires a shorter period compared to the conventional process, so that there is residual alpha-amylase carried into the fermentation process. In one embodiment, the improved liquefaction process results in an increased yield of ethanol using commercially available alpha-amylases.

### 1. DEFINITIONS & ABBREVIATIONS

### 1.1. Definitions

As used herein, “starch” refers to any material comprised of the complex polysaccharide carbohydrates of plants, comprised of amylose and amylopectin with the formula (C₆H₁₀O₅)ₓ, wherein X can be any number. In particular, the term refers to any plant-based material including but not limited to grains, grasses, tubers, and roots and more specifically wheat, barley, corn, rye, oats, sorgum, milo, rice, sorghum, brans, cassaya, millet, potato, sweet potato, and tapioca.

“Alpha-amylase” (e.g., E.C. 3.2.1.1) generally refers to enzymes that catalyze the hydrolysis of alpha-1,4-glucosidic linkages. These enzymes have also been described as those effecting the exo- or endohydrolysis of 1,4-α-D-glucosidic linkages in polysaccharides containing 1,4-α-linked D-glucose units. For the purpose of the present disclosure, “alpha-amylases” refers to those enzymes having relatively high thermostability, i.e., with sustained activity at higher temperatures, e.g., above 80° C. Accordingly, alpha-amylases are capable of liquefying starch, which is performed at a temperature above 80° C.

“Alpha-amylase unit” (AAU) refers to alpha-amylase activity measured according to the method disclosed in U.S. Pat. No. 5,958,739, which is incorporated herein by reference. In brief, the assay uses p-nitrophenyl maltoheptoside (PNP-G₇) as the substrate with the non-reducing terminal sugar chemically blocked. PNP-G₇ can be cleaved by an endo-amylase, for example alpha-amylase. Following the cleavage, an alpha-glucosidase and a glucoamylase digest the substrate to liberate free PNP molecules, which display a yellow color and can be measured by visible spectophometry at 410 nm. The rate of PNP release is proportional to alpha-amylase activity. The AAU of a given sample is calculated against a standard control. One unit of AAU refers to the amount of enzyme required to hydrolyze 10 mg of starch per minute under specified conditions.

As used herein, “residual alpha-amylase activity” refers to the portion, i.e., 10% or more, of the initial alpha-amylase that remains enzymatically active after the completion of the liquefaction step(s).

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell, or express native genes that are otherwise abnormally expressed, under expressed or not expressed at all.

The terms “protein” and “polypeptide” are used interchangeably herein.

The conventional one-letter or three-letter code for amino acid residues is used herein.

A “signal sequence” means a sequence of amino acids bound to the N-terminal portion of a protein, which facilitates the secretion of the mature form of the protein outside the cell. The definition of a signal sequence is a functional one. The mature form of the extracellular protein lacks the signal sequence that is cleaved off during the secretion process.

A “gene” refers to a DNA segment that is involved in producing a polypeptide and includes regions preceding and following the coding regions as well as intervening sequences (introns) between individual coding segments (exons).

The term “nucleic acid” encompasses DNA, RNA, single stranded or double stranded and chemical modifications thereof. The terms “nucleic acid” and “polynucleotide” may be used interchangeably herein.

A “vector” refers to a polynucleotide sequence designed to introduce nucleic acids into one or more cell types, wherein the elements of the vector are operably linked. Vectors include cloning vectors, expression vectors, shuttle vectors, plasmids, phage particles, cassettes and the like.

An “expression vector” as used herein means a DNA construct comprising a DNA sequence that is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences that control termination of transcription and translation.

A “promoter” is a regulatory sequence that is involved in binding RNA polymerase to initiate transcription of a gene. The promoter may be an inducible promoter or a constitutive promoter.

“Under transcriptional control” is a term well understood in the art that indicates that transcription of a polynucleotide sequence, usually a DNA sequence, depends on its being operably linked to an element that contributes to the initiation of, or promotes transcription.

“Under translational control” is a term well understood in the art that indicates a regulatory process that occurs after mRNA has been formed.

As used herein, when describing proteins and genes that encode them, the term for the gene is italicized, (e.g., the gene that encodes amyL (B. licheniformis AA) may be denoted as amyL). The term for the protein is generally not italicized and the first letter is generally capitalized (e.g., the protein encoded by the amyL gene may be denoted as AmyL or amyL).

The term “derived” encompasses the terms “originated from,” “obtained” or “obtainable from,” and “isolated from.”

The term “operably linked” refers to juxtaposition wherein the elements are in an arrangement allowing them to be functionally related. For example, a promoter is operably linked to a coding sequence if it controls the transcription of the sequence.

The term “selective marker” refers to a gene capable of expression in a host that allows for ease of selection of those hosts containing an introduced nucleic acid or vector. Examples of selectable markers include but are not limited to antimicrobials (e.g., hygromycin, bleomycin, or chloramphenicol) and/or genes that confer a metabolic advantage, such as a nutritional advantage on the host cell.

A polynucleotide or a polypeptide having a certain percent (e.g., about 80%, about 85%, about 90%, about 95%, or about 99%) of sequence identity with another sequence means that, when aligned, that percentage of bases or amino acid residues are the same in comparing the two sequences. This alignment and the percent identity can be determined using any suitable software program known in the art, for example those described in CURRENT PROTOCOLS IN MOLECULAR BIOLOGY, Ausubel et al., eds., 1987, Supplement 30, section 7.7.18. Representative programs include the Vector NTI Advance™ 9.0 (Invitrogen Corp. Carlsbad, Calif.), GCG Pileup, FASTA (Pearson et al. (1988) Proc. Nat'l Acad. Sci. USA 85:2444-2448), and BLAST (BLAST Manual, Altschul et al., Nat'l Cent. Biotechnol. Inf., Nat'l Lib. Med. (NCIB NLM NIH), Bethesda, Md., and Altschul et al., (1997) Nucleic Acids Res. 25:3389-3402) programs. Another typical alignment program is ALIGN Plus (Scientific and Educational Software, PA), generally using default parameters. Another sequence software program that finds use is the TFASTA Data Searching Program available in the Sequence Software Package Version 6.0 (Genetics Computer Group, University of Wisconsin, Madison, Wis.).

The term “parent” or “parent sequence” refers to a sequence that is native or naturally occurring in a host cell. Parent sequences include, but are not limited to, the sequences of Bacillus licheniformis alpha-amylase LAT (U.S. Ser. No. 12/263,804, filed Nov. 3, 2008) and Geobacillus stearothermophilus alpha-amylase (U.S. Ser. No. 12/263,886, filed Nov. 3, 2008).

“Variants” may have at least about 45%, at least about 50%, at least about 55%, at least about 60%, at least about 65%, at least about 70%, at least about 75%, at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, or at least about 99.5% sequence identity to a polypeptide sequence when optimally aligned for comparison.

The term “property” or grammatical equivalents thereof in the context of a polypeptide, as used herein, refers to any characteristic or attribute of a polypeptide that can be selected or detected. These properties include, but are not limited to oxidative stability, substrate specificity, catalytic activity, thermal stability, pH activity profile, resistance to proteolytic degradation, K_{M}, k_{CAT}, k_{CAT}/K_{M} ratio, protein folding, ability to bind a substrate and ability to be secreted.

“Thermostable” or “thermostability” means the enzyme retains active after exposure to elevated temperatures. The thermostability of an alpha-amylase is evaluated by its half-life (t_{1/2}), where half of the enzyme activity is lost at a given temperature. The half-life is measured by determining the specific alpha-amylase activity of the enzyme remaining over time at a given temperature, particularly at a temperature used for a specific application, e.g., liquefaction.

“Host strain” or “host cell” means a suitable host for an expression vector or DNA construct comprising a polynucleotide encoding a variant alpha-amylase enzyme according to the present disclosure. Specifically, host strains are typically bacterial cells. In a typical embodiment, “host cell” means both the cells and protoplasts created from the cells of a microbial strain and particularly a Bacillus sp.

The term “culturing” refers to growing a population of microbial cells under suitable conditions in a liquid or solid medium. In one embodiment, culturing refers to fermentative bioconversion of a starch substrate containing granular starch to an end product (typically in a vessel or reactor). Fermentation is the enzymatic and anaerobic breakdown of organic substances by microorganisms to produce simpler organic compounds. While fermentation occurs under anaerobic conditions it is not intended that the term be solely limited to strict anaerobic conditions, as fermentation also occurs in the presence of oxygen.

The term “contacting” refers to the placing of the respective enzyme(s) in sufficiently close proximity to the respective substrate to enable the enzyme(s) to convert the substrate to the end product. Those skilled in the art will recognize that mixing solutions of the enzyme with the respective substrates can effect contacting.

The term “enzymatic conversion” in general refers to the modification of a substrate by enzyme action. The term as used herein also refers to the modification of a starch substrate by the action of an enzyme.

As used herein, “saccharification” refers to enzymatic conversion of starch to glucose.

“Gelatinization” means solubilization of a starch molecule by cooking to form a viscous suspension.

“Liquefaction” refers to the stage in starch conversion in which gelatinized starch is hydrolyzed to give low molecular weight soluble dextrins.

The term “degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP>3 denotes polymers with a degree of polymerization of greater than 3.

The terms “end product” or “desired end product” refer to any carbon-source derived molecule product that is enzymatically converted from the starch substrate.

As used herein the term “dry solids content (ds)” refers to the total solids of a slurry in % on a dry weight basis.

The term “slurry” refers to an aqueous mixture containing insoluble solids.

The term “residual starch” refers to the amount of starch present in grain by-products after fermentation. Typically, the amount of residual starch present in 100 gram of distillers dried grain with solubles (DDGS) may be one of the parameters to evaluate the efficiency of starch utilization in an ethanol production process.

As used herein, “a recycling step” refers to the recycling of mash components, which may include residual starch, enzymes and/or microorganisms to ferment substrates comprising starch.

The term “mash” refers to a mixture of a fermentable carbon source (carbohydrate) in water used to produce a fermented product, such as an alcohol. In some embodiments, the term “beer” and “mash” are used interchangeability.

The term “stillage” means a mixture of non-fermented solids and water, which is the residue after removal of alcohol from a fermented mash.

The terms “distillers dried grain (DDG)” and “distillers dried grain with solubles (DDGS)” refer to a useful by-product of grain fermentation.

As used herein, “ethanologenic microorganism” refers to a microorganism with the ability to convert a sugar or oligosaccharide to ethanol. The ethanologenic microorganisms are ethanologenic by virtue of their ability to express one or more enzymes that individually or together convert sugar to ethanol.

As used herein, “ethanol producer” or “ethanol producing microorganism” refers to any organism or cell that is capable of producing ethanol from a hexose or pentose. Generally, ethanol-producing cells contain an alcohol dehydrogenase and a pyruvate decarboxylase. Examples of ethanol producing microorganisms include fungal microorganisms such as yeast. The typical yeast used in ethanol production includes species and strains of Saccharomyces, e.g., S. cerevisiae.

The term “heterologous” with reference to a polynucleotide or protein refers to a polynucleotide or protein that does not naturally occur in a host cell. In some embodiments, the protein is a commercially important industrial protein. It is intended that the term encompass proteins that are encoded by naturally occurring genes, mutated genes, and/or synthetic genes.

The term “endogenous” with reference to a polynucleotide or protein refers to a polynucleotide or protein that occurs naturally in the host cell.

The terms “recovered,” “isolated,” and “separated” as used herein refer to a compound, protein, cell, nucleic acid or amino acid that is removed from at least one component with which it is naturally associated.

As used herein, “transformed,” “stably transformed” and “transgenic” used in reference to a cell means the cell has a non-native (e.g., heterologous) nucleic acid sequence integrated into its genome or as an episomal plasmid that is maintained through multiple generations.

As used herein, “expression” refers to the process by which a polypeptide is produced based on the nucleic acid sequence of a gene. The process includes both transcription and translation.

The term “introduced” in the context of inserting a nucleic acid sequence into a cell, means “transfection,” or “transformation” or “transduction” and includes reference to the incorporation of a nucleic acid sequence into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed (e.g., transfected mRNA).

As used herein, “specific activity” means an enzyme unit defined as the number of moles of substrate converted to product by an enzyme preparation per unit time under specific conditions. Specific activity is expressed as units (U)/mg of protein.

The term “yield” with reference to the ethanol yield refers to the relative efficiency of ethanol production from starting materials, e.g., corn. In one embodiment, the ethanol yield is calculated as “gal UD/bushel corn,” reflecting gallon of undenatured ethanol produced per bushel of corn. A bushel of corn weighs about 56 pounds.

“ATCC” refers to American Type Culture Collection located at Manassas, Va. 20108 (ATCC).

“NRRL” refers to the Agricultural Research Service Culture Collection, National Center for Agricultural Utilization Research (and previously known as USDA Northern Regional Research Laboratory), Peoria, Ill.

“A,” “an,” and “the” include plural references unless the context clearly dictates otherwise.

As used herein, “comprising” and its cognates are used in their inclusive sense; that is, equivalent to the term “including” and its corresponding cognates.

### 1.2. Abbreviations

The following abbreviations apply unless indicated otherwise:

AA alpha-amylase

AAU alpha-amylase unit

AGU glucoamylase activity unit

AOS α-olefinsulfonate

AS alcohol sulfate

BAA bacterial alpha-amylase

cDNA complementary DNA

CMC carboxymethylcellulose

DDG distillers dried grains

DDGS distillers dried grain with solubles

DE Dextrose Equivalent

DNA deoxyribonucleic acid

DNS 3,5-dinitrosalicylic acid

DP3 degree of polymerization with three subunits

DPn degree of polymerization with n subunits

DS, ds dry solid

DSC differential scanning calorimetry

DTMPA diethyltriaminepentaacetic acid

EC enzyme commission for enzyme classification

EDTA ethylenediaminetetraacetic acid

EDTMPA ethylenediaminetetramethylene phosphonic acid

EO ethylene oxide

F&HC fabric and household care

g gram

gal gallon

HFCS high fructose corn syrup

HFSS high fructose starch based syrup

IPTG isopropyl β-D-thiogalactoside

LAS linear alkylbenezenesulfonate

LU Lipase Units

MES 2-(N-morpholino)ethanesulfonic acid

MM gpy million gallon per year

MW molecular weight

nm nanometer

NOBS nonanoyloxybenzenesulfonate

NTA nitrilotriacetic acid

PCR polymerase chain reaction

PEG polyethyleneglycol

pI isoelectric point

PNP-G₇ p-nitrophenyl maltoheptoside

ppm parts per million

PVA poly(vinyl alcohol)

PVP poly(vinylpyrrolidone)

RAU Reference Amylase Units

RMS root mean square

RNA ribonucleic acid

rpm revolutions per minute

SAS secondary alkane sulfonates

1×SSC 0.15 M NaCl, 0.015 M sodium citrate, pH 7.0

SSF simultaneous saccharification and fermentation

TAED tetraacetylethylenediamine

TNBS trinitrobenzenesulfonic acid

w/v weight/volume

w/w weight/weight

wt wild-type

UD undenatured

μL microliter

### 2. ETHANOL PRODUCTION FROM STARCH

In general, alcohol (ethanol) production from starch can be separated into four steps: milling, liquefaction, saccharification, and fermentation.

2.1. Raw Materials

In the starch processing of the present disclosure, particularly in the ethanol processes of the present disclosure, the starting raw material is typically whole grain or at least mainly whole grain. The raw material may be chosen from a wide variety of starch-containing whole grain crops including corn, milo, potato, cassaya, sorghum, wheat, and barley. In one embodiment, the starch-containing raw material is whole grain selected from the group consisting of corn, milo, potato, cassaya, sorghum, wheat, and barley, or any combinations thereof. In a typical embodiment, the starch-containing raw material is whole grain selected from the group consisting of corn, wheat, and barley, or any combinations thereof.

2.2. Milling

The grain is milled in order to open up the structure and allow for further processing. Three commonly used processes are wet willing, dry milling, and various fractionation schemes. In dry milling, the whole kernel is milled and used in the subsequent steps of the process. On the other hand, wet milling gives a very good separation of germ and meal (starch granules and protein), so that it is usually applied, with a few exceptions, at locations where there is a parallel production of syrups. The various fractionation processes, as variations of the wet or dry milling processes, involve varying degrees of separation of the various components. Most ethanol comes from dry milling. Alternatively, the starch to be processed may be a highly refined starch quality, for example, at least about 90%, at least about 95%, at least about 97%, or at least about 99.5% pure.

2.3. Gelatinization and Liquefaction

As used herein, the term “liquefaction” or “liquefy” means a process by which starch is converted to less viscous and shorter chain dextrins. This process involves gelatinization of starch simultaneously with or followed by the addition of an alpha-amylase.

In some embodiments, the starch substrate prepared as described above is slurried with water. The starch slurry may contain starch as a weight percent of dry solids of about 10-55%, about 20-45%, about 30-45%, about 30-40%, or about 30-35%. To optimize alpha-amylase stability and activity, the pH of the slurry may be adjusted.

In one aspect, the alpha-amylase can be used at a higher dose than the one that is normally required in conventional liquefaction processes. The alpha-amylase dose for the improved liquefaction may be at least about 1.7, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, or about 10.0 times the dose required to sufficiently reduce the viscosity of the mash in a liquefaction processes performed at a temperature of about 85° C. Typically, an alpha-amylase is dosed in a range of 2-10 AAU/g ds to sufficiently reduce the viscosity of the mash, i.e., reaching a DE value of at least about 10 within 90 minutes, in a liquefaction processes performed at a temperature of about 85° C. Representative alpha-amylases used in liquefaction processes include GC 358 and SPEZYME® XTRA (Danisco US Inc., Genencor Division), and LIQUOZYME® SC and LIQUOZYME® SC DS (Novozymes A/S, Denmark). Alternatively, alpha-amylase products including but not limited to SPEZYME® FRED, SPEZYME® HPA, Maxalig™ ONE (Danisco US Inc., Genencor Division) and FUELZYME® LF (Verenium Corp.) can be used in the process. Blends of any of the above mentioned enzyme products may also be used.

The slurry of starch is herein liquefied at a temperature lower than about 99° C., e.g., in a range of about 70° C. to about 95° C., about 80° C. to about 95° C., about 85° C. to about 95° C., and optionally about 88° C. to about 92° C. The liquefaction may last about 30-300 minutes, e.g., about 30-180 minutes. The conventional high temperature treatment, e.g., jet-cooking, which is typically performed at a temperature between about 100-125° C., is purposely omitted in the presently disclosed methods. The omission of the high temperature treatment results in the presence of a residual alpha-amylase activity after liquefying. The residual alpha-amylase activity may be at least 10% or about 15%.

2.4. Saccharification

Following liquefaction, the mash is further hydrolyzed through saccharification to produce low molecular sugars (DP 1-2) that are can be readily metabolized by yeast. During saccharification, the hydrolysis is generally accomplished enzymatically by the presence of a glucoamylase. Typically, an alpha-glucosidase and/or an acid alpha-amylase may also be supplemented in addition of the glucoamylase.

A full saccharification step may typically last up to about 72 hours. In some embodiments, the saccharification step and fermentation step are combined and the process is referred to as simultaneous saccharification and fermentation (SSF) or simultaneous saccharification, yeast propagation and fermentation. In some embodiments, a pre-saccharification step of 1-4 hours may be included between the liquefaction step and the saccharification step.

2.5. Fermentation

The organism used in fermentations will depend on the desired end product. Typically, if ethanol is the desired end product, yeast will be used as the fermenting organism. In some embodiments, the ethanol-producing microorganism is a yeast, and specifically Saccharomyces, such as strains of S. cerevisiae (U.S. Pat. No. 4,316,956). A variety of S. cerevisiae are commercially available and these include but are not limited to Ethanol Red™ (Fermentis), THERMOSACC® and Superstart™ (Lallemand Ethanol Technology), FALI (Fleischmann's Yeast), FERMIOL® (DSM Specialties), Bio-Ferm® XR(NACB), and Angel alcohol yeast (Angel Yeast Company, China). The amount of starter yeast employed in the methods is an amount effective to produce a commercially significant amount of ethanol in a suitable amount of time, (e.g. to produce at least 10% ethanol from a substrate having between 25-40% ds in less than 72 hours). Yeast cells can be supplied in amounts of about 10⁴ to 10¹², and typically from about 10⁷ to 10¹° viable yeast count per ml of fermentation broth. The fermentation will include in addition to a fermenting microorganisms (e.g., yeast), nutrients, optionally additional enzymes, including but not limited to phytases. The use of yeast in fermentation is well known. See, e.g., THE ALCOHOL TEXTBOOK, K. A. JACQUES ET AL., EDS. 2003, NOTTINGHAM UNIVERSITY PRESS, UK.

The improved liquefaction method as described herein may result in an improved ethanol yield. The improved ethanol yield is about 1.0%, about 1.5%, about 2.0%, about 2.5%, or about 3.0%, more than that of an ethanol production process having starch liquefied at a temperature of about 85° C. and at a alpha-amylase dosage required to reach a DE value of at least about 10 within 90 minutes. The ethanol yield may be expressed as “gal UD/bushel corn,” reflecting gallon of undenatured ethanol produced per bushel corn. Modern technologies typically allow for an ethanol yield in the range of about 2.5 to about 2.8 gal UD/bushel corn. See Bothast & Schlicher, “Biotechnological Processes for Conversion of Corn into Ethanol,” Appl. Microbiol. Biotechnol. 67: 19-25 (2005). The improved ethanol production efficiency may attribute to more efficient starch utilization in the starch processing as described herein. At the end of ethanol production, the residual starch present in 100 gram of grain by-products is at least about 10%, about 20%, or about 30% lower than that of an ethanol production process having starch liquefied at a temperature of about 85° C. and at a alpha-amylase dosage required to reach a DE value of at least about 10 within 90 minutes.

In further embodiments, by use of appropriate fermenting microorganisms as known in the art, the fermentation end product may include without limitation glycerol, 1,3-propanediol, gluconate, 2-keto-D-gluconate, 2,5-diketo-D-gluconate, 2-keto-L-gulonic acid, succinic acid, lactic acid, amino acids and derivatives thereof. More specifically, when lactic acid is the desired end product, a Lactobacillus sp. (L. casei) may be used; when glycerol or 1,3-propanediol are the desired end products, E. coli may be used; and when 2-keto-D-gluconate, 2,5-diketo-D-gluconate, and 2-keto-L-gulonic acid are the desired end products, Pantoea citrea may be used as the fermenting microorganism. The above enumerated list are only examples and one skilled in the art will be aware of a number of fermenting microorganisms that may be appropriately used to obtain a desired end product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

2.6. Distillation

Optionally, following fermentation, ethanol may be extracted by, for example, distillation and optionally followed by one or more process steps. In some embodiments, the yield of ethanol produced by the present methods will be at least about 8%, at least about 10%, at least about 12%, at least about 14%, at least about 15%, at least about 16%, at least about 17%, at least about 18%, and at least about 23% v/v. The ethanol obtained according to the process of the present disclosure may be used as, for example, fuel ethanol, drinking ethanol, i.e., potable neutral spirits, or industrial ethanol.

2.7. By-Products

Grain by-products from the fermentation typically are used for animal feed in either a liquid form or a dried form. If the starch is wet milled, non-starch by-products include crude protein, oil, and fiber, e.g., corn gluten meal. If the starch is dry-milled, the by-products may include animal feed co-products, such as distillers' dried grains (DDG) and distillers' dried grain plus solubles (DDGS). When the grain is dry milled and mixed in a slurry before liquefaction and saccharification, however, no grain is left as a by-product.

### 3. ENZYMES INVOLVED IN ETHANOL PRODUCTION FROM STARCH

### 3.1. Alpha-Amylases

Alpha-amylases as described in the present disclosure are those displaying relatively high thermostability and thus capable of liquefying starch at a temperature above 80° C. Alpha-amylases suitable for the liquefaction process may be from fungal or bacterial origin, particularly alpha-amylases isolated from thermophilic bacteria, such as Bacillus species. These Bacillus alpha-amylases are commonly referred to as “Termamyl-like alpha-amylases.” Well-known Termamyl-like alpha-amylases include those isolated from B. licheniformis, B. amyloliquefaciens, and Geobacillus stearothermophilus (previously known as Bacillus stearothermophilus). Other Termamyl-like alpha-amylases include those derived from Bacillus sp. NCIB 12289, NCIB 12512, NCIB 12513, and DSM 9375, all of which are described in detail in WO 95/26397 and incorporated herein by reference. Contemplated alpha-amylases may also derive from Aspergillus species, e.g., A. oryzae and A. niger alpha-amylases. In addition, commercially available alpha-amylases and products containing alpha-amylases include TERMAMYL™ SC, FUNGAMYL™, LIQUOZYME® SC and SAN™ SUPER (Novozymes A/S, Denmark), and SPEZYME® XTRA, GC 358, SPEZYME® FRED, SPEZYME® FRED-L, and SPEZYME® HPA (Danisco US Inc, Genencor Division).

In one aspect, alpha-amylases may be wild-type parent enzymes. In another aspect, the alpha-amylase may be a variant of the parent enzyme. In another aspect, the variant alpha-amylase may have about 50%, about 55%, about 60%, about 65%, about 70%, about 75%, about 80%, about 85%, about 90%, about 95%, or about 99% sequence identity to a Termamyl-like alpha-amylase. In another aspect, the variant alpha-amylase may have about 50%, about 55%, about 60%, about 65%, about 70%, about 75%, about 80%, about 85%, about 90%, about 95%, or about 99% sequence identity to Bacillus licheniformis alpha-amylase LAT (U.S. Ser. No. 12/263,804, filed Nov. 3, 2008) or Geobacillus stearothermophilus AmyS alpha-amylase (U.S. Ser. No. 12/263,886, filed Nov. 3, 2008). Contemplated variants are described in WO 96/23874, WO 97/41213, and WO 99/19467, and include the Geobacillus stearothermophilus alpha-amylase variant, alpha-amylase TTC, having the mutations Δ(181-182)+N193F compared to the wild-type alpha-amylase disclosed in WO99/19467.

In some embodiments, a variant alpha-amylase may display one or more altered properties compared to those of the parent enzyme. The altered properties may advantageously enable the variant alpha-amylase to perform effectively in liquefaction. Similarly, the altered properties may result in improved performance of the variant compared to its parent. These properties may include substrate specificity, substrate binding, substrate cleavage pattern, thermal stability, pH/activity profile, pH/stability profile, stability towards oxidation, stability at lower levels of calcium ion (Ca²⁺), and/or specific activity. Representative alpha-amylase variants, which can be useful in the present disclosure, include, but are not limited to those described in US 2008/0220476, published Sep. 11, 2008; US 2008/0160573, published Jul. 3, 2008; US 2008/0153733, published Jun. 26, 2008; US 2008/0083406, published Apr. 10, 2008; U.S. Ser. No. 12/263,804, filed Nov. 3, 2008; and U.S. Ser. No. 12/263,886, filed Nov. 3, 2008; all of which are incorporated herein by reference.

In yet another aspect, the liquefaction may involve the use of a blend of at least two alpha-amylases, each of with may display different properties. The blend may further comprise a phytase.

Alpha-amylase activity may be determined according to the method disclosed in U.S. Pat. No. 5,958,739, with minor modifications. In brief, the assay uses p-nitrophenyl maltoheptoside (PNP-G₇) as the substrate with the non-reducing terminal sugar chemically blocked. PNP-G₇ can be cleaved by an endo-amylase, for example alpha-amylase. Following the cleavage, an alpha-glucosidase and a glucoamylase digest the substrate to liberate free PNP molecules, which display a yellow color and can be measured by visible spectophometry at 410 nm. The rate of PNP release is proportional to alpha-amylase activity. The alpha-amylase activity of a sample is calculated against a standard control.

Several methods for introducing mutations into genes are known in the art. The DNA sequence encoding a parent α-amylase may be isolated from any cell or microorganism producing the α-amylase in question, using various methods well known in the art. First, a genomic DNA and/or cDNA library should be constructed using chromosomal DNA or messenger RNA from the organism that produces the α-amylase to be studied. Then, if the amino acid sequence of the α-amylase is known, homologous, labeled oligonucleotide probes may be synthesized and used to identify alpha-amylase-encoding clones from a genomic library prepared from the organism in question. Alternatively, a labeled oligonucleotide probe containing sequences homologous to a known α-amylase gene could be used as a probe to identify α-amylase-encoding clones, using hybridization and washing conditions of lower stringency.

Yet another method for identifying alpha-amylase-encoding clones would involve inserting fragments of genomic DNA into an expression vector, such as a plasmid, transforming α-amylase-negative bacteria with the resulting genomic DNA library, and then plating the transformed bacteria onto agar containing a substrate for α-amylase, thereby allowing clones expressing the α-amylase to be identified.

Alternatively, the DNA sequence encoding the enzyme may be prepared synthetically by established standard methods, e.g., the phosphoamidite method described by S. L. Beaucage and M. H. Caruthers, Tetrahedron Letters 22: 1859-1869 (1981) or the method described by Matthes et al., EMBO J. 3: 801-895 (1984). In the phosphoamidite method, oligonucleotides are synthesized, e.g., in an automatic DNA synthesizer, purified, annealed, ligated and cloned in appropriate vectors.

Finally, the DNA sequence may be of mixed genomic and synthetic origin, mixed synthetic and cDNA origin or mixed genomic and cDNA origin, prepared by ligating fragments of synthetic, genomic or cDNA origin (as appropriate, the fragments corresponding to various parts of the entire DNA sequence), in accordance with standard techniques. The DNA sequence may also be prepared by polymerase chain reaction (PCR) using specific primers, for instance as described in U.S. Pat. No. 4,683,202 or R. K. Saiki et al., Science 239: 487-491 (1988).

Once an α-amylase-encoding DNA sequence has been isolated, and desirable sites for mutation identified, mutations may be introduced using synthetic oligonucleotides. These oligonucleotides contain nucleotide sequences flanking the desired mutation sites; mutant nucleotides are inserted during oligonucleotide synthesis. In a specific method, a single-stranded gap of DNA, bridging the α-amylase-encoding sequence, is created in a vector carrying the α-amylase gene. Then the synthetic nucleotide, bearing the desired mutation, is annealed to a homologous portion of the single-stranded DNA. The remaining gap is then filled in with DNA polymerase I (Klenow fragment) and the construct is ligated using T4 DNA ligase. A specific example of this method is described in Morinaga et al., Biotechnology 2: 636-639 (1984). U.S. Pat. No. 4,760,025 discloses the introduction of oligonucleotides encoding multiple mutations by performing minor alterations of the cassette. An even greater variety of mutations can be introduced at any one time by the Morinaga method, because a multitude of oligonucleotides, of various lengths, can be introduced.

Another method of introducing mutations into α-amylase-encoding DNA sequences is described in Nelson and Long, Analytical Biochem. 180: 147-151 (1989). It involves the 3-step generation of a PCR fragment containing the desired mutation introduced by using a chemically synthesized DNA strand as one of the primers in the PCR reactions. From the PCR-generated fragment, a DNA fragment carrying the mutation may be isolated by cleavage with restriction endonucleases and reinserted into an expression plasmid.

Alternative methods for providing variants of the disclosure include gene shuffling, e.g., as described in WO 95/22625 (from Affymax Technologies N.V.) or in WO 96/00343 (from Novo Nordisk A/S), or other corresponding techniques resulting in a hybrid enzyme comprising the desired mutation(s), e.g., substitution(s) and/or deletion(s).

A DNA sequence encoding the wild-type alpha-amylase or the variant produced by methods described above, or by any alternative methods known in the art, can be expressed, in enzyme form, using an expression vector that typically includes control sequences encoding a promoter, operator, ribosome binding site, translation initiation signal, and, optionally, a repressor gene or various activator genes.

The recombinant expression vector carrying the DNA sequence encoding a wild-type alpha-amylase or a variant may be any vector, as long as it is conveniently subjected to recombinant DNA procedures. The choice of vector will often depend on the host cell into which it is to be introduced. The vector may be an autonomously replicating vector, i.e., a vector that exists as an extrachromosomal entity, the replication of which is independent of chromosomal replication, e.g., a plasmid, a bacteriophage, or an extrachromosomal element, minichromosome or an artificial chromosome. Alternatively, the vector may be one that, when introduced into a host cell, is integrated into the host cell genome and replicated together with the chromosome(s) into which it has been integrated.

In the vector, the DNA sequence should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the DNA sequence encoding an alpha-amylase variant of the present disclosure, especially in a bacterial host, are the promoter of the lac operon of E. coli, the Streptomyces coelicolor agarase gene dagA promoters, the promoters of the Bacillus licheniformis alpha-amylase gene (amyL), the promoters of the Geobacillus stearothermophilus maltogenic amylase gene (amyM), the promoters of the Bacillus amyloliquefaciens alpha-amylase (amyQ), the promoters of the Bacillus subtilis xylA and xylB genes, etc. For directing the transcription in a fungal host, examples of useful promoters are those derived from the gene encoding A. oryzae TAKA amylase, Rhizomucor miehei aspartic proteinase, A. niger neutral alpha-amylase, A. niger acid stable alpha-amylase, A. niger glucoamylase, Rhizomucor miehei lipase, A. oryzae alkaline protease, A. oryzae triose phosphate isomerase, and A. nidulans acetamidase.

The expression vector may also comprise a suitable transcription terminator and, in eukaryotes, polyadenylation sequences operably connected to the DNA sequence encoding the alpha-amylase variant of the present disclosure. Termination and polyadenylation sequences may suitably be derived from the same sources as the promoter.

The vector may further comprise a DNA sequence enabling the vector to replicate in the host cell in question. Examples of such sequences are the origins of replication of plasmids pUC19, pACYC177, pUB110, pE194, pAMB1 and pIJ702.

The vector may also comprise a selectable marker, e.g., a gene the product of which complements a defect in the host cell, such as the dal genes from B. subtilis or B. licheniformis, or one that confers antibiotic resistance, such as ampicillin, kanamycin, chloramphenicol or tetracyclin resistance. Furthermore, the vector may comprise Aspergillus selection markers such as amdS, argB, niaD and sC, a marker giving rise to hygromycin resistance, or the selection may be accomplished by co-transformation, e.g., as described in WO 91/17243.

While intracellular expression may be advantageous in some respects, e.g., when using certain bacteria as host cells, it is generally favored that the expression is extracellular. In general, the Bacillus alpha-amylases mentioned herein comprise a pre-region permitting secretion of the expressed protease into the culture medium. If desirable, this pre-region may be replaced by a different pre-region or signal sequence, conveniently accomplished by substitution of the DNA sequences encoding the respective pre-regions.

The procedures used to ligate the DNA construct encoding an alpha-amylase variant, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2nd Ed., Cold Spring Harbor, 1989).

The cell of the present disclosure, comprising either a DNA construct or an expression vector as defined above, is advantageously used as a host cell in the recombinant production of an alpha-amylase variant of the present disclosure. The cell may be transformed with the DNA construct encoding the variant, conveniently by integrating the DNA construct (in one or more copies) in the host chromosome. This integration is generally considered to be an advantage as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

The cell of the present disclosure may be a cell of a higher organism such as a mammal or an insect, but is typically a microbial cell, e.g., a bacterial or a fungal (including yeast) cell. Examples of suitable bacteria are gram-positive bacteria such as Bacillus subtilis, Bacillus licheniformis, Bacillus lentus, Bacillus brevis, Geobacillus stearothermophilus, Bacillus alkalophilus, Bacillus amyloliquefaciens, Bacillus coagulans, Bacillus circulans, Bacillus lautus, Bacillus megaterium, Bacillus thuringiensis, or Streptomyces lividans or S. murinus, or gram-negative bacteria such as E. coli. The transformation of the bacteria may, for instance, be effected by protoplast transformation or by using competent cells in a manner known per se.

The yeast organism may favorably be selected from a species of Saccharomyces or Schizosaccharomyces, e.g., Saccharomyces cerevisiae. The filamentous fungus may advantageously belong to a species of Aspergillus, e.g., Aspergillus oryzae or A. niger. Fungal cells may be transformed by a process involving protoplast formation and transformation of the protoplasts followed by regeneration of the cell wall in a manner known per se. A suitable procedure for transformation of Aspergillus host cells is described in EP 238 023.

An alpha-amylase variant may be further produced by cultivating a host cell as described above under conditions conducive to the production of the variant and recovering the variant from the cells and/or culture medium. The medium used to cultivate the cells may be any conventional medium suitable for growing the host cell in question and obtaining expression of the alpha-amylase variant. Suitable media are available from commercial suppliers or may be prepared according to published recipes (e.g., as described in catalogues of the American Type Culture Collection). The alpha-amylase variant secreted from the host cells may conveniently be recovered from the culture medium by well-known procedures. For example, after the cells are separated from the medium by centrifugation or filtration, the proteinaceous components of the medium are removed by precipitating using a salt such as ammonium sulfate. The alpha-amylase or its variant is further purified by the use of chromatographic procedures such as ion exchange chromatography, affinity chromatography, or the like.

### 3.2. Glucoamylases

Another enzyme contemplated for use in the starch processing, especially during saccharification, is a glucoamylase (EC 3.2.1.3). Glucoamylases are commonly derived from a microorganism or a plant. For example, glucoamylases can be of fungal or bacterial origin.

Exemplary fungal glucoamylases are Aspergillus glucoamylases, in particular A. niger G1 or G2 glucoamylase (Boel et al. (1984), EMBO J. 3(5): 1097-1102), or variants thereof, such as disclosed in WO 92/00381 and WO 00/04136; A. awamori glucoamylase (WO 84/02921); A. oryzae glucoamylase (Agric. Biol. Chem. (1991), 55(4): 941-949), or variants or fragments thereof. Other contemplated Aspergillus glucoamylase variants include variants with enhanced thermal stability: G137A and G139A (Chen et al. (1996), Prot. Eng. 9: 499-505); D257E and D293E/Q (Chen et al. (1995), Prot. Eng. 8: 575-582); N182 (Chen et al. (1994), Biochem. J. 301: 275-281); disulphide bonds, A246C (Fierobe et al. (1996), Biochemistry, 35: 8698-8704); and introduction of Pro residues in positions A435 and S436 (Li et al. (1997) Protein Eng. 10: 1199-1204).

Exemplary fungal glucoamylases may also include Trichoderma reesei glucoamylase and its homologs as disclosed in U.S. Pat. No. 7,413,879 (Danisco US Inc., Genencor Division). These glucoamylases include T. reesei glucoamylase, Hypocrea citrina var. americana glucoamylase, Hypocrea vinosa glucoamylase, Trichoderma sp. glucoamylase, Hypocrea gelatinosa glucoamylase, Hypocrea orientalis glucoamylase, Trichoderma konilangbra glucoamylase, Trichoderma sp. glucoamylase, Trichoderma harzianum glucoamylase, Trichoderma longibrachiatum glucoamylase, Trichoderma asperellum glucoamylase, and Trichoderma strictipilis glucoamylase.

Other contemplated glucoamylases include Talaromyces glucoamylases, in particular derived from T. emersonii (WO 99/28448), T. leycettanus (U.S. Pat. No. RE 32,153), T. duponti, or T. thermophilus (U.S. Pat. No. 4,587,215). Contemplated bacterial glucoamylases include glucoamylases from the genus Clostridium, in particular C. thermoamylolyticum (EP 135138) and C. thermohydrosulfuricum (WO 86/01831).

Suitable glucoamylases include the glucoamylases derived from Aspergillus oryzae, such as a glucoamylase having about 50%, about 55%, about 60%, about 65%, about 70%, about 75%, about 80%, about 85%, or even about 90% identity to the amino acid sequence shown in WO 00/04136. Suitable glucoamylases may also include the glucoamylases derived from Trichoderma reesei, such as a glucoamylase having about 50%, about 55%, about 60%, about 65%, about 70%, about 75%, about 80%, about 85%, or even about 90% identity to the amino acid sequence shown in WO 08/045,489 (Danisco US Inc., Genencor Division). T. reesei glucoamylase variants with altered properties, such as those disclosed in WO 08/045,489 and U.S. Ser. No. 12/292,563, filed Nov. 20, 2008 (Danisco US Inc., Genencor Division), may be particularly useful.

Also suitable are commercial glucoamylases, such as Spirizyme® Fuel, Spirizyme® Plus, and Spirizyme® Ultra (Novozymes A/S, Denmark), G-ZYME® 480, G-ZYME® 480 Ethanol, GC 147, DISTILLASE®, and FERMENZYME® (Danisco US Inc., Genencor Division). Glucoamylases may be added in an amount of about 0.02-2.0 AGU/g ds or about 0.1-1.0 AGU/g ds, e.g., about 0.2 AGU/g ds.

### 3.3. Phytases

Phytases are useful for the present disclosure as they are capable of hydrolyzing phytic acid under the defined conditions of the incubation and liquefaction steps. In some embodiments, the phytase is capable of liberating at least one inorganic phosphate from an inositol hexaphosphate (phytic acid). Phytases can be grouped according to their preference for a specific position of the phosphate ester group on the phytate molecule at which hydrolysis is initiated (e.g., as 3-phytases (EC 3.1.3.8) or as 6-phytases (EC 3.1.3.26)). A typical example of phytase is myo-inositol-hexakiphosphate-3-phosphohydrolase.

Phytases can be obtained from microorganisms such as fungal and/or bacterial organisms. Some of these microorganisms include e.g. Aspergillus (e.g., A. niger, A. terreus, A. ficum and A. fumigatus), Myceliophthora (M. thermophila), Talaromyces (T. thermophilus) Trichoderma spp (T. reesei). and Thermomyces (WO 99/49740). Phytases are also available from Penicillium species, e.g., P. hordei (ATCC No. 22053), P. piceum (ATCC No. 10519), or P. brevi-compactum (ATCC No. 48944). See, e.g., U.S. Pat. No. 6,475,762. In addition, phytases are available from Bacillus (e.g., B. subtilis, Pseudomonas, Peniophora, E. coli, Citrobacter, Enterbacter, and Buttiauxella (see WO2006/043178)).

Commercial phytases are available such as NATUPHOS (BASF), RONOZYME P (Novozymes A/S), PHZYME (Danisco A/S, Diversa), and FINASE (AB Enzymes). The Maxalig™ ONE (Danisco US Inc., Genencor Division) blend contains a thermostable phytase that is capable of efficiently reducing viscosity of the liquefact and breaking down phytic acid. The method for determining microbial phytase activity and the definition of a phytase unit has been published by Engelen et al. (1994) J. of AOAC Int., 77: 760-764. The phytase may be a wild-type phytase, a variant, or a fragment thereof.

In one embodiment, the phytase is one derived from the bacterium Buttiauxiella spp. The Buttiauxiella spp. includes B. agrestis, B. brennerae, B. ferragutiase, B. gaviniae, B. izardii, B. noackiae, and B. warmboldiae. Strains of Buttiauxella species are available from DSMZ, the German National Resource Center for Biological Material (Inhoffenstrabe 7B, 38124 Braunschweig, Germany). Buttiauxella sp. strain P1-29 deposited under accession number NCIMB 41248 is an example of a particularly useful strain from which a phytase may be obtained and used according to the present disclosure. In some embodiments, the phytase is BP-wild-type, a variant thereof (such as BP-11) disclosed in WO 06/043178, or a variant as disclosed in US 2008/0220498, published Sep. 11, 2008. For example, a BP-wild-type and variants thereof are disclosed in Table 1 of WO 06/043178.

### 3.4. Other Enzymes

Another aspect contemplates the additional use of a beta-amylase. Beta-amylases (EC 3.2.1.2) are exo-acting maltogenic amylases, which catalyze the hydrolysis of 1,4-α-glucosidic linkages into amylose, amylopectin, and related glucose polymers, thereby releasing maltose. Beta-amylases have been isolated from various plants and microorganisms (Fogarty et al., PROGRESS IN INDUSTRIAL MICROBIOLOGY, Vol. 15, pp. 112-115, 1979). These beta-amylases are characterized by having optimum temperatures in the range from about 40° C. to about 65° C., and optimum pH in the range from about 4.5 to about 7.0. Contemplated β-amylases include, but are not limited to, beta-amylases from barley Spezyme® BBA 1500, Spezyme® DBA, Optimalt™ ME, Optimalt™ BBA (Danisco US Inc, Genencor Division); and Novozym™ WBA (Novozymes A/S).

Another enzyme that can optionally be added is a debranching enzyme, such as an isoamylase (EC 3.2.1.68) or a pullulanases (EC 3.2.1.41). Isoamylase hydrolyses α-1,6-D-glucosidic branch linkages in amylopectin and β-limit dextrins and can be distinguished from pullulanases by the inability of isoamylase to attack pullulan and by the limited action of isoamylase on α-limit dextrins. Debranching enzymes may be added in effective amounts well known to the person skilled in the art.

### EXAMPLES

The following examples are not to be interpreted as limiting, but are exemplary means of using the methods disclosed.

### Example 1

### Comparison of Ethanol Production

Ethanol production from raw material was performed using (1) the conventional liquefaction method that includes the high-temperature jet-cooking, and (2) the modified liquefaction method that omits high temperature treatment and increases the alpha-amylase dose. Under the conventional liquefaction method, a slurry at pH 5.9 was treated at 81° C. for 28 minutes using 1.2 AAU/g ds GC 358 (Danisco US Inc., Genencor Division). Subsequently, the slurry was jet-cooked at 103° C. for 3 minutes. After cooling, an additional 2.6 AAU/g ds GC 358 was added, and the slurry was further treated at 84° C. for 144 minutes.

Under the modified liquefaction method, a slurry at pH 5.8 was treated at 86° C. for 29 minutes using 8.3 AAU/g ds GC 358 (Danisco US Inc., Genencor Division). Instead of jet-cooking, the slurry was further treated at 85° C. for 144 minutes without additional alpha-amylase.

A simultaneous saccharification fermentation (SSF) process was used to produce ethanol. Briefly, a 27° Brix mash was fermented using 1-5×10⁸ yeast cells (Bio-Ferm® XR (NACB)) per ml of mash in the presence of 150-300 ppm urea. The glucoamylase, GC 147 (Danisco US Inc., Genencor Division), was supplemented at 0.6 GAU/g ds. The fermentation was conducted at 31-33° C. and at an average pH of about 4.5, and lasted 50-70 hours.

Based on measurements over an eight-month period, the average ethanol yield was 2.753 gal UD/bushel corn using the conventional liquefaction method. The standard deviation was 0.033. When the improved liquefaction method was used over the next five-month period, however, the ethanol yield increased to 2.827 gal UD/bushel corn on average with a standard deviation of 0.021 corn on average. A comparison between the conventional liquefaction method and the improved liquefaction method is present in Table 1. A statistical analysis using a two-tailed t-test indicated that the two yields are different at a 99.5% confidence level (P<0.005).

TABLE 1
Ethanol production comparison.

| | | Conventional | Improved |
|---|---|---|---|
| | | liquefaction | liquefaction |
| slurry pH | | 5.9 | 5.8 |
| Liquefaction | temperature (° C.) | 81 | 86 |
| conditions | time (min) | 28 | 29 |
| | alpha-amylase | 1.2 | 8.3 |
| | (AAU/g ds) | | |
| Jet-cooking | temperature (° C.) | 103 | omitted |
| conditions | time (min) | 3 | |
| Liquefaction | temperature (° C.) | 84 | 85 |
| conditions | time (min) | 144 | 144 |
| | additional | 2.6 | 0.0 |
| | alpha-amylase | | |
| | (AAU/g ds) | | |

| | | |
|---|---|---|
| Average ethanol yield | 2.753 | 2.827 |
| (gal UD/bushel corn) | | |
| Standard deviation | 0.033 | 0.021 |

In addition, the presently described liquefaction process was compared with a conventional liquefaction process that was performed at a lower temperature. The parameters of the processes and the results were presented in Table 2. The average ethanol yield from the improved liquefaction was 2.77 gal UD/bushel corn, while that from the conventional low-temperature liquefaction was 2.73 gal UD/bushel corn. A statistical analysis using a two-tailed t-test indicated that the two yields are different at a 97.5% confidence level (P≦0.025). The improved liquefaction method, therefore, is able to produce more ethanol form starch.

TABLE 2
Ethanol production comparison.

| | | Conventional | Improved |
|---|---|---|---|
| | | liquefaction | liquefaction |
| slurry pH | | 6.07 | 6.11 |
| Liquefaction | temperature | 82.1 | 83.2 |
| conditions | (° C.) | | |
| | time (min) | 44 | 56 |
| | alpha- | 1.13 | 5.85 |
| | amylase | | |
| | (AAU/g ds) | | |
| Jet-cooking | temperature | 82.6 | 84.8 |
| conditions | (° C.) | | |
| | time (min) | 9.4 | 10.8 |
| Liquefaction | temperature | 80.6 | 83.3 |
| conditions | (° C.) | | |
| | time (min) | 170 | 222 |
| | additional | 2.23 | 0 |
| | alpha- | | |
| | amylase | | |
| | (AAU/g ds) | | |

| | | |
|---|---|---|
| Average ethanol yield | 2.73 | 2.77 |
| (gal UD/bushel corn) | (28 Fermentors) | (13 Fermentors) |
| Standard deviation | 0.066 | 0.050 |

### Example 2

### Comparison of Residual Starch in DDSG

The observed increase of ethanol production by the modified liquefaction method may be attributed to a more efficient liquefaction of starch. To test this hypothesis, the residual starch was measured in DDGS that are resulted from different liquefaction treatments as shown in Example 1. Specifically, two commercially available alpha-amylases, GC 358 (Danisco US Inc., Genencor Division) and LIQUOZYME® SC DS (Novozymes A/S, Denmark), were used.

To determine the amount of residual starch, the sample is first subject to liquefaction with an alpha-amylase followed by saccharification with a glucoamylase. The resulting glucose is then used to calculate the amount residual starch present in the sample.

Starting from DDGS, a grind was produced using a Falling Mill, so that it could pass through a 20 mesh screen. To determine the amount of soluble glucose present in DDGS, 2 gram of the sample was mixed with 50 mL distilled water in a 100 mL Kohlrausch flask. The sample was incubated at room temperature with constant stirring for 1 hour. Subsequently, 1.0 mL 1N H₂SO₄ was added, and the total volume was brought up to 100 mL with distilled water. The sample was then filtered using a 0.2 μm syringe filter and subject to HPLC analysis to determine the amount of soluble glucose present.

To measure the amount of residual starch present in DDGS, 2 grams of sample was mixed with 45 mL MOPS buffer, pH 7.0 (supplemented with 5 mM calcium chloride) in a 100 mL Kohlrausch flask. One milliliter of a 1:50 dilution of SPEZYME® FRED was added. The flask was covered with foil and placed with donut weights in a pan of boiling water for 15 minutes. An addition 1 mL of a 1:50 dilution of SPEZYME® FRED was added. The flask was kept in a 95° C. water bath for 45 minutes, then a 60° C. water bath for 1 hour. The pH was adjusted by adding 20 ml of acetate buffer, pH 4.2. Then, 1.0 ml of a 1:100 dilution of OPTIDEX® L-400 (Danisco A/S) was added, and the saccharification was performed at 60° C. for 18 hours. Saccharification was stopped by boiling the sample for 15 minutes and then cooling it to room temperature. One milliliter of H₂SO₄ was added and the total volume was brought up to 100 mL with distilled water. The sample was then filtered using a 0.2 μm syringe filter and subject to HPLC analysis. The amount of glucose in the sample after liquefaction and saccharification was determined by comparing to a 0.5% glucose standard made in 0.01 N sulfuric acid. The amount of soluble glucose was deducted from the amount of total glucose to obtain the amount of glucose derived from the residual starch. The amount of glucose derived from the residual starch was then converted to the amount of residual starch by a multiplication factor of 0.9, which reflects the difference between glucose and starch molecules.

As shown in Table 3, a significantly lower level of residual starch in DDGS was observed in fermentations employing the improved liquefaction process, indicating that starch is more efficiently converted. The data thus suggests that the improved liquefaction process, by omitting the higher temperature treatment while increasing the alpha-amylase dose, enables residual alpha-amylase activity to be carried into the fermentation step. The carryover of residual alpha-amylase is likely responsible for more efficient starch utilization and increased ethanol production.

TABLE 3
Comparison of the amount of residual starch in DDGS

| | Relative | | % Residual |
|---|---|---|---|
| | Enzyme | | Starch in |
| Enzyme Type | Dose^{#} | Jet-cooking | DDGS |
| GC358 | 1× | YES | 5.09 |
| | 1× | NO | 3.41 |
| | 2× | NO | 3.16 |
| LIQUOZYME ® | 2× | NO | 3.74 |
| SC DS | 4× | NO | 3.54 |

^{#}1× alpha-amylase dose is equivalent of 3.8 AAU/g ds.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A method of starch processing comprising liquefying a starch in the presence of an alpha-amylase at a temperature no higher than 99° C., wherein the alpha-amylase is dosed at least 1.7 times of an amount that is required to reach a DE value of at least about 10 within 90 minutes in a liquefaction process performed at a temperature of about 85° C.

2. The method of claim 1, wherein the starch is liquefied at a temperature in a range of about 70 to about 95° C.

3. The method of claim 1, wherein the starch is liquefied at a temperature in a range of about 80 to about 95° C.

4. The method of claim 1, wherein the starch is liquefied at a temperature in a range of about 85 to about 95° C.

5. The method of claim 1, wherein the starch is liquefied at a temperature in a range of about 88° C. to about 92° C.

6. The method of claim 1, wherein liquefying is performed for about 30-300 minutes.

7. The method of claim 1, wherein liquefying is performed for about 30-180 minutes.

8. The method of claim 1, wherein residual alpha-amylase activity of at least about 10% is present after liquefying.

9. The method of claim 1, wherein residual alpha-amylase activity of at least about 15% is present after liquefying.

10. The method of claim 1 further comprising saccharifying the starch that is liquefied.

11. The method of claim 1 further comprising fermenting the starch to produce ethanol.

12. The method of claim 11 further comprising recovering the ethanol.

13. The method of claim 11 further comprising distilling the starch to obtain the ethanol, wherein fermenting and distilling are carried out simultaneously, separately, or sequentially.

14. The method of claim 11, wherein residual starch present in 100 gram of grain by-products at the end of ethanol production is at least 10% lower than that of an ethanol production process having starch liquefied at a temperature of about 85° C. and at a alpha-amylase dosage required to reach a DE value of at least about 10 within 90 minutes.

15. The method of claim 11, wherein the method is capable of producing ethanol at a yield at least about 1.0% higher than that by an ethanol production process having starch liquefied at a temperature of about 85° C. and at a alpha-amylase dosage required to reach a DE value of at least about 10 within 90 minutes.

16. The method of claim 1, wherein starch is from a group consisting of corns, cobs, wheat, barley, rye, milo, and potatoes, and any combination of these.

17. The method of claim 1, wherein the starch is from corn or corn mash.
